(19) United States
(12) Patent Application Publication
(10) Pub. No.: US 2006/0173064 A1
(43) Pub. Date: Aug. 3, 2006

(54) (-)-1-(3,4-Dichlorophenyl)-3-azabi cyclo[3.1.0]hexane, compositions thereof, and uses for treating alcohol-related disorders

(76) Inventors: Arnold S. Lippa, Ridgewood, NJ (US); Anthony Basile, Hoboken, NJ (US); Joseph W. Epstein, Monroe, NY (US)

Correspondence Address: GRAYBEAL JACKSON HALEY LLP;Suite 350, 155 - 108th Avenue NE, Bellevue, WA, 98004-5901, US

(21) Appl. No.: 11/254,242

(22) Filed: Oct. 18, 2005

Related U.S. Application Data
(62) Division of application No. 10/764,371, filed on Jan. 23, 2004.
Application No. 10/764,371 is a division of application No. 10/425,545, filed on Apr. 29, 2003, now Pat. No. 6,716,868.
Application No. 10/425,545 is a division of application No. 09/939,071, filed on Aug. 24, 2001, now Pat. No. 6,569,887.

(51) Int. Cl. C07D 209/52; A61K 31/403

(52) U.S. Cl. 514/412; 548/452

(57) ABSTRACT

The present invention relates to (−)-1-(3,4-dichlorophenyl )-3-azabicyclo[3.1.0]hexane and pharmaceutically acceptable salts thereof, compositions comprising (−)-1-(3,4-dichlorophenyl)-3-azabicyclo[3.1.0]hexane or a pharmaceutically acceptable salt thereof, and methods for treating or preventing a disorder alleviated by inhibiting dopamine reuptake. In certain embodiments the methods and compositions of the invention are effective for treating attention-deficit disorder, depression, obesity, Parkinson's disease, a tic disorder, and/or an addictive disorder. In more detailed embodiments, methods and compositions of the invention are provided for treating an alcohol-related addictive disorder, for example alcohol abuse, alcohol dependence, excess alcohol consumption, and/or alcohol withdrawal. The (−)-1-(3,4-dichlorophenyl)-3-azabicyclo[3.1.0]hexane or pharmaceutically acceptable salt thereof may be employed within the methods and compositions of the invention in a form or composition that is substantially free of its corresponding (+)-enantiomer.

### CROSS-REFERENCES TO RELATED APPLICATIONS

This application is a continuation-in-part of application Ser. No. 10/764,371 filed Jan. 23, 2004, which is a divisional of application Ser. No. 10/425,545 filed on Apr. 29, 2003, now U.S. Pat. No. 6,716,868 dated Apr. 6, 2004, which is a divisional of application Ser. No. 09/939,071 filed on Aug. 24, 2001, now U.S. Pat. No. 6,569,887 dated May 27, 2003.

### FIELD OF THE INVENTION

The present invention relates to (−)-1-(3,4-dichlorophenyl)-3-azabicyclo[3.1.0]hexane and pharmaceutically acceptable salts thereof, compositions comprising (−)-1-(3,4-dichlorophenyl)-3-azabicyclo[3.1.0]hexane or a pharmaceutically acceptable salt thereof and methods for treating or preventing a disorder alleviated by inhibiting dopamine reuptake comprising administering to a patient (−)-1-(3,4dichlorophenyl)-3-azabicyclo[3.1.0]hexane or a pharmaceutically acceptable salt thereof, including alcohol-related disorders.

### BACKGROUND OF THE INVENTION

Dopamine is a monoamine neurotransmitter that plays a critical role in the function of the hypothalamic-pituitary-adrenal axis and in the integration of information in sensory, limbic, and motor systems. The primary mechanism for termination of dopamine neurotransmission is through reuptake of released dopamine by Na⁺/Cl⁻ dependent plasma membrane transporters (Hoffman et al., 1998, Front. Neuroendocrinol. 19(3):187-231). Depending on the surrounding ionic conditions, the dopamine transporter can function as a mediator of both inward directed dopamine transport (i.e., “reuptake”) and outward directed dopamine transport (i.e., “release”). The functional significance of the dopamine transporter is its regulation of dopamine neurotransmission by terminating the action of dopamine in a synapse via reuptake (Hitri et al., 1994, Clin. Pharmacol. 17:1-22).

Attention deficit disorder is a learning disorder involving developmentally inappropriate inattention with or without hyperactivity. The primary signs of attention deficit disorder are a patient's inattention and impulsivity. Inappropriate inattention causes increased rates of activity or reluctance to participate or respond. A patient suffering from attention deficit disorder exhibits a consistent pattern of inattention and/or hyperactivity-impulsivity that is more frequent and severe than is typically observed in individuals at a comparable level of development. (See, e.g., U.S. Pat. No. 6,121,261 to Glatt et al.).

Patients having Parkinson's disease display jittery movements of the limbs, head, and jaw. Parkinson's disease is associated with bradykinesia, rigidity and falling (Stacy et al., 1996, Am. Fam. Phys. 53:1281-1287). The movement disturbances observed in Parkinson's disease patients result from degeneration of dopamine neurons, loss of nerve terminals, and dopamine deficiency. It is hypothesized that the cause of the degeneration of the dopamine neurons results from apoptosis resulting from increased levels of cytokines (Nagatsu et al., 2000, J Neural Transm. Suppl. 60:277-290). Abnormalities in the dopamine transporter have been implicated in Parkinson's disease (Hitri et al., 1994, Clin. Neuropharmacol. 17:1-22). Symptoms of Parkinson's disease can be attenuated by compounds like pergolide which mimics the actions of dopamine or by compounds that inhibit dopamine metabolism (e.g., carbidopa) or by dopamine precursors (e.g., L-DOPA±carbidopa).

Appetite suppression is a reduction, a decrease or, in cases of excessive food consumption, an amelioration in appetite. This suppression reduces the desire or craving for food. Appetite suppression can result in weight loss or weight control as desired. Appetite suppression can regulate food intake through drug administration directed to one or more systems known to play a role in food digestion. See, for example, Sullivan et al., “Mechanisms of Appetite Modulation By Drugs,” Federation Proceedings, Volume 44, No. 1, Part 1, pages 139-144 (1985). Methods for controlling appetite suppression include the regulation of serotonin level, thermogenesis and the inhibition of lipogenesis. (See e.g., U.S. Pat. No. 5,911,992 to Braswell et al.).

Depression is one of the most common of the mental illnesses, having a morbidity rate of over 10% in the general population. Depression is characterized by feelings of intense sadness, despair, mental slowing, loss of concentration, pessimistic worry, agitation, and self-deprecation (Harrison's Principles of Internal Medicine 2490-2497 (Fauci et al. eds., 14th ed. 1998)). Depression can have physical manifestations including insomnia, hypersomnia, anorexia, weight loss, overeating, decreased energy, decreased libido, and disruption of normal circadian rhythms of activity, body temperature, and endocrine functions. In fact, as many as 10% to 15% of depressed individuals display suicidal behavior. R. I. Baldessarini, Drugs and the Treatment of Psychiatric Disorders: Depression and Mania, in Goodman and Gilman 's The Pharmacological Basis of Therapeutics 431 (9th ed. 1996). Anhedonia is one of the principal (core) symptoms of depression. Dopamine pathways have been linked to pleasure seeking behaviors, and strategies to increase synaptic concentrations of dopamine have been proposed as antidepressant therapies. (See e.g., D'Aquila et al., 2000, Eur. J Pharmacal. 405:365-373).

Obesity is commonly referred to as a condition of increased body weight due to excessive fat. Drugs to treat obesity can be divided into three groups: (1) those that decrease food intake, such as drugs that interfere with monoamine receptors, such as noradrenergic receptors, serotonin receptors, dopamine receptors, and histamine receptors; (2) those that increase metabolism; and (3) those that increase thermogenesis or decrease fat absorption by inhibiting pancreatic lipase (Bray, 2000, Nutrition 16:953-960 and Leonhardt et al., 1999, Eur. J Nutr. 38:1-13).

Many drugs can cause physical and/or psychological addiction. Those most well known drugs include opiates, such as heroin, opium and morphine; sympathomimetics, including cocaine and amphetamines; sedative-hypnotics, including alcohol, benzodiazepines and barbiturates; and nicotine, which has effects similar to opioids and sympathomimetics. Drug addiction is characterized by a craving or compulsion for taking the drug and an inability to limit its intake. Additionally, drug dependence is associated with drug tolerance, the loss of effect of the drug following repeated administration, and withdrawal, the appearance of physical and behavioral symptoms when the drug is not consumed. Sensitization occurs if repeated administration of a drug leads to an increased response to each dose. Tolerance, sensitization, and withdrawal are phenomena evidencing a change in the central nervous system resulting from continued use of the drug. This change motivates the addicted individual to continue consuming the drug despite serious social, legal, physical and/or professional consequences. (See, e.g., U.S. Pat. No. 6,109,269 to Rise et al.). Cocaine addiction remains one of the major health problems in the United States. Fundamental studies from many laboratories have shown that cocaine blocks the uptake of dopamine from the synaptic cleft of the dopamine transporter (Kreek, 1996, J Addict. Dis. 15:73-96). For example, the inhibition action of cocaine on reuptake of released dopamine, however, does not fully explain the development and maintenance of addictive behavior. Coexistence of functionally antagonistic, inhibition actions of cocaine on the dopamine release and reuptake of the released dopamine might be responsible for fluctuations in dopamine transmission (Kiyatkin, 1994, Int. J Neurosci. 78:75-101).

Certain pharmaceutical agents have been administered for treating addiction. U.S. Pat. No. 5,556,838 to Mayer et al. discloses the use of nontoxic NMDA-blocking agents co-administered with an addictive substance to prevent the development of tolerance or withdrawal symptoms. U.S. Pat. No. 5,574,052 to Rose et al. discloses coadministration of an addictive substance with an antagonist to partially block the pharmacological effects of the substance. U.S. Pat. No. 5,075,341 to Mendelson et al. discloses the use of a mixed opiate agonist/antagonist to treat cocaine and opiate addiction. U.S. Pat. No. 5,232,934 to Downs discloses administration of 3-phenoxypyridine to treat addiction. U.S. Pat. Nos. 5,039,680 and 5,198,459 to Imperato et al. disclose using a serotonin antagonist to treat chemical addiction. U.S. Pat. No. 5,556,837 to Nestler et. al. discloses infusing BDNF or NT-4 growth factors to inhibit or reverse neurological adaptive changes that correlate with behavioral changes in an addicted individual. U.S. Pat. No. 5,762,925 to Sagan discloses implanting encapsulated adrenal medullary cells into a patient's central nervous system to inhibit the development of opioid intolerance. Bupropion has dopamine reuptake inhibition properties and is used to treat nicotine addiction.

Dopaminergic reward pathways have been implicated in disorders resulting from addictive behaviors. Variants of the dopamine D2 receptor gene have been associated with alcoholism, obesity, pathological gambling, attention deficit hyperactivity disorder, Tourette syndrome, cocaine dependence, nicotine dependence, polysubstance abuse, and other drug dependency (Noble, 1994, Alcohol Supp. 2:35-43 and Blum et al., 1995, Pharmacogenetics 5:121-141). Since reduced dopaminergic functions have been found in individuals with a minor Al allele of the dopamine D2 receptor, it has been suggested that the dopamine D2 receptor may be a reinforcement or reward gene (Noble, 1994, Alcohol Supp. 2:35-43). Furthermore, several studies suggest that an associate of dopamine D2 receptor gene polymorphisms are associated with impulsive-addictive-compulsive behavior, i.e., “Reward Deficiency Syndrome” (reviewed by Blum et al., 1995, Pharmacogenetics 5:121-141).

U.S. Pat. No. 4,435,419 to Epstein et al. discloses racemic, (±)-1-(3,4-dichlorophenyl)-3-azabicyclo[3.1.0]hexane for use as an anti-depressant agent.

U.S. Pat. No. 6,204,284 to Beer et al. discloses racemic, (±)-1-(3,4-dichlorophenyl)-3-azabicyclo[3.1.0]hexane for use in the prevention or relief of a withdrawal syndrome resulting from addition to drugs and for the treatment of chemical dependencies.

Administration of a racemic, i.e., 50:50, mixture of the (+)- and the (−)-enantiomer of any drug, for example (±)-1-(3,4-dichlorophenyl)-3-azabicyclo[3.1.0]hexane, to a patient can be disadvantageous. First, the racemic mixture might be less pharmacologically active than one of its enantiomers, rendering racemic drugs inherently inefficient. Second, the racemic mixture may be more toxic to a patient than one of its enantiomers, so that administration of a racemic mixture can lead to undesirable side effects in a patient.

Accordingly, there is a clear need in the art for an enantiomer, the enantiomer being preferably substantially free of the corresponding opposite enantiomer, which would overcome one or both of the aforementioned disadvantages.

### SUMMARY OF THE INVENTION

In one embodiment, the invention provides (−)-1-(3,4-dichlorophenyl)-3-azabicyclo[3.1.0]hexane and pharmaceutically acceptable salts thereof. (−)-1-(3,4-dichlorophenyl)-3-azabicyclo[3.1.0]hexane and pharmaceutically acceptable salts thereof are useful for treating or preventing a disorder alleviated by inhibiting dopamine reuptake.

The present invention further provides compositions comprising an effective amount of (−)-1-(3,4-dichlorophenyl)-3-azabicyclo[3.1.0]hexane or a pharmaceutically acceptable salt thereof. The present compositions can additionally comprise a pharmaceutically acceptable vehicle. These compositions are useful for treating or preventing a disorder alleviated by inhibiting dopamine reuptake.

The present invention also provides pharmaceutical compositions for treating or preventing ethanol consumption in a patient comprising an effective amount of (−)-1-(3,4-dichlorophenyl)-3-azabicyclo[3.1.0]hexane or a pharmaceutically acceptable salt thereof.

The present invention additionally provides pharmaceutical compositions for treating or preventing ethanol consumption and depression in a patient comprising an effective amount of (−)-1-(3,4-dichlorophenyl)-3-azabicyclo[3.1.0]hexane or a pharmaceutically acceptable salt thereof.

In another embodiment, the invention provides a method for treating or preventing a disorder alleviated by inhibiting dopamine reuptake, comprising administering to a patient in need of such treatment or prevention an effective amount of (−)-1-(3,4-dichlorophenyl)-3-azabicyclo[3.1.0]hexane or a pharmaceutically acceptable salt thereof.

In still another embodiment, the invention provides a method for treating or preventing attention-deficit disorder, depression, obesity, Parkinson's disease, a tic disorder, or an addictive disorder, comprising administering to a patient in need of such treatment or prevention an effective amount of (−)-1-(3,4-dichlorophenyl)-3-azabicyclo[3.1.0]hexane or a pharmaceutically acceptable salt thereof.

In another embodiment, the invention provides a method for treating or preventing ethanol consumption, comprising administering to a patient in need of such treatment or prevention an effective amount of (−)1-(3,4-dichlorophenyl)-3-azabicyclo[3.1.0]hexane or a pharmaceutically acceptable salt thereof.

In a further embodiment, the invention provides a method for treating or preventing ethanol consumption and depression, comprising administering to a patient in need of such treatment or prevention an effective amount of (−)1-(3,4-dichlorophenyl)-3-azabicyclo[3.1.0]hexane or a pharmaceutically acceptable salt thereof.

Preferably, (−)-1-(3,4-dichlorophenyl)-3-azabicyclo[3.1.0]hexane or a pharmaceutically acceptable salt thereof, particularly when used in the present methods or compositions, is substantially free of its corresponding (+)-enantiomer. In exemplary embodiments, (−)-1-(3,4-dichlorophenyl)-3-azabicyclo[3.1.0]hexane or a pharmaceutically acceptable salt thereof substantially free of its corresponding (+)-enantiomer is used to treat or prevent a disorder alleviated by selectively inhibiting dopamine uptake. Use according to these embodiments, surprisingly and advantageously does not block norepinephrine or serotonin transport, in particular, norepinephrine or serotonin uptake. It has unexpectedly been discovered that use of (−)-1-(3,4-dichlorophenyl)-3-azabicyclo[3.1.0]hexane or a pharmaceutically acceptable salt thereof substantially free of its corresponding (+) enantiomer to treat or prevent a disorder alleviated by inhibiting dopamine uptake avoids side effects such as cardiovascular effects, sleep interruption, hypertension or sexual dysfunction associated with norepinephrine or serotonin uptake inhibitors.

In still another embodiment, the invention provides a method for obtaining (−)-1-(3,4-dichlorophenyl)-3-azabicyclo[3.1.0]hexane substantially free of its corresponding (+)-enantiomer, comprising the steps of:

(a) passing a solution of an organic eluent and (±)-1-(3,4-dichlorophenyl)-3-azabicyclo[3.1.0]hexane over a chiral polysaccharide stationary phase to provide a first fraction containing (−)-1-(3,4-dichlorophenyl)-3-azabicyclo[3.1.0]hexane; and

(b) passing the first fraction over the chiral polysaccharide stationary phase to provide a second fraction containing (−)-1-(3,4-dichlorophenyl)-3azabicyclo[3.1.0]hexane substantially free of its corresponding (+)-enantiomer.

In still another embodiment, the invention provides a method for obtaining (−)-1-(3,4-dichlorophenyl)-3-azabicyclo[3.1.0]hexane substantially free of its corresponding (+)-enantiomer, comprising the steps of:

(a) passing a solution of an organic eluent and (±)-1-(3,4-dichlorophenyl)-3-azabicyclo[3.1.0]hexane over a chiral polysaccharide stationary phase to provide a first fraction containing (−)-1-(3,4-dichlorophenyl)-3-azabicyclo[3.1.0]hexane;

(b) concentrating the first fraction to provide a residue; and

(c) passing a solution of an organic eluent and the residue over a chiral polysaccharide stationary phase to provide a second fraction containing (−)-1-(3,4 dichlorophenyl)-3-azabicyclo[3.1.0]hexane substantially free of its corresponding (+)-enantiomer.

The present invention may be understood more fully by reference to the detailed description and examples, which are intended to exemplify non-limiting embodiments of the invention.

### 4. DETAILED DESCRIPTION OF THE INVENTION

### 4.1. DEFINITIONS

The term “substantially free of its corresponding (+)-enantiomer” means containing no more than about 5% w/w of the corresponding (+)-enantiomer, preferably no more than about 2% w/w of the corresponding (+)-enantiomer, more preferably no more than about 1% w/w of the corresponding (+)-enantiomer.

The term “corresponding (+)-enantiomer” when used in connection with (−)-1-(3,4-dichlorophenyl)-3-azabicyclo[3.1.0]hexane or a pharmaceutically acceptable salt thereof means “(+)-1-(3,4 dichlorophenyl)-3-azabicyclo[3.1.0]hexane” or a pharmaceutically acceptable salt thereof.

A “patient” is an animal, including, but not limited to, an animal such a cow, monkey, horse, sheep, pig, chicken, turkey, quail, cat, dog, mouse, rat, rabbit, and guinea pig, and is more preferably a mammal, and most preferably a human.

The phrase “pharmaceutically acceptable salt” as used herein is a salt formed from an acid and the basic nitrogen group of (−)-1-(3,4-dichlorophenyl)-3-azabicyclo[3.1.0]hexane. Exemplary salts include, but are not limited, to sulfate, citrate, acetate, oxalate, chloride, bromide, iodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1-methylene-bis-(2-hydroxy-3-naphthoate)) salts.

### 4.2 (−)-1-(3,4-DICHLOROPHENYL)-3-AZABICYCLO[3.1.0]HEXANE

(−)-1-(3,4-dichlorophenyl)-3-azabicyclo[3.1.0]hexane, preferably that substantially free of its corresponding (+)-enantiomer, can be obtained from (±)-1-(3,4-dichlorophenyl)-3-azabicyclo[3.1.0]hexane using chiral chromatographic methods, such as high-performance liquid chromatography (“HPLC”) with a suitable, e.g., chiral, column. (±)-1-(3,4-dichlorophenyl)-3-azabicyclo[3.1.0]hexane is obtainable using methods disclosed in U.S. Pat. No. 4,435,419 to Epstein et al.

In an exemplary embodiment, (−)-1-(3,4-dichlorophenyl)-3-azabicyclo[3.1.0]hexane is obtained by passing a solution of an organic eluent and (+)-1-(3,4-dichlorophenyl)-3-azabicyclo[3.1.0]hexane over a chiral polysaccharide stationary phase. In more detailed embodiments, the polysaccharide is starch or a starch derivative. Advantageously, the chiral stationary phase is within a chiral HPLC column, for example, a CHIRALPAK AD column manufactured by Daicel and commercially available from Chiral Technologies, Inc., Exton, Pa., more preferably a 1 cm×25 cm CHIRALPAK AD HPLC column. An exemplary eluent is a hydrocarbon solvent adjusted in polarity with a miscible polar organic solvent. In more detailed embodiments, the organic eluent contains a non-polar, hydrocarbon solvent present in about 95% to about 99.5% (volume/volume) and a polar organic solvent present in about 5 to about 0.5% (volume/volume). In other detailed embodiments, the hydrocarbon solvent is hexane and the miscible polar organic solvent is isopropylamine.

Passing the solution of the organic eluent and (±)-1-(3,4-dichlorophenyl)-3-azabicyclo[3.1.0]hexane over the chiral polysaccharide stationary phase provides a first fraction (i.e., one or more fractions) containing (−)-1-(3,4-dichlorophenyl)-3-azabicyclo[3.1.0]hexane. The first fraction can be directly passed over the chiral polysaccharide stationary phase to provide a second fraction (i.e., one or more fractions) containing (−)-1-(3,4-dichlorophenyl)-3-azabicyclo[3.1.0]hexane substantially free of its corresponding (+)-enantiomer. Alternatively, the first fraction can be concentrated to provide a residue that can be diluted with an organic eluent, and the resulting solution can be passed over the chiral polysaccharide stationary phase to provide a second fraction containing (−)-1-(3,4-dichlorophenyl)-3-azabicyclo[3.1.0]hexane substantially free of its corresponding (+)-enantiomer. Either way, the second fraction(s) can be concentrated, for example in vacuo, to obtain a solid form of (−)-1-(3,4-dichlorophenyl)-3-azabicyclo[3.1.0]hexane substantially free of its corresponding (+)-enantiomer.

### 4.3 THERAPEUTIC USES OF (−)-1-(3,4-DICHLOROPHENYL)-3-AZABICYCLO[3.1.0]HEXANE

In accordance with the invention, (−)-1-(3,4-dichlorophenyl)-3-azabicyclo[3.1.0]hexane or a pharmaceutically acceptable salt thereof is administered to a patient, preferably a mammal, more preferably a human, for the treatment or prevention of a disorder alleviated by inhibiting dopamine reuptake. In one embodiment, “treatment” or “treating” refers to an amelioration of a disorder alleviated by inhibiting dopamine reuptake, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disorder alleviated by inhibiting dopamine reuptake, either physically, e.g., normalization of a discernible symptom, physiologically, e.g., normalization of a physical parameter, or both. In yet another embodiment, “treatment” or “treating” refers to delaying the onset of a disorder alleviated by inhibiting dopamine reuptake.

In certain embodiments, (−)-1-(3,4-dichlorophenyl)-3-azabicyclo[3.1.0]hexane or a pharmaceutically acceptable salt thereof is administered to a patient, preferably a mammal, more preferably a human, as a preventative measure against acquiring a disorder alleviated by inhibiting dopamine reuptake. As used herein, “prevention” or “preventing” refers to a reduction of the risk of acquiring a disorder alleviated by inhibiting dopamine reuptake or to the reduction of the risk of recurrence of the disorder once cured or restored to a normal state. In one embodiment, (−)-1-(3,4 dichlorophenyl)-3-azabicyclo[3.1.0]hexane or a pharmaceutically acceptable salt thereof is administered as a preventative measure to a patient. According to this embodiment, the patient can have a genetic predisposition to a disorder alleviated by inhibiting dopamine reuptake, such as a family history of biochemical imbalance in the brain, or a non-genetic predisposition to a disorder alleviated by inhibiting dopamine reuptake. Accordingly, the (−) 1-(3,4-dichlorophenyl)-3-azabicyclo[3.1.0]hexane and pharmaceutically acceptable salts thereof can be used for the treatment of one manifestation of a disorder alleviated by inhibiting dopamine reuptake and prevention of another.

### 4.3.1 Disorders Alleviated Using (−)-1-(3,4-Dichlorophenyl)-3-Azabicyclo[3.1.0]Hexane

(−)-1-(3,4-dichlorophcnyl)-3-azabicyclo[3.1.0]hexane and pharmaceutically acceptable salts thereof are useful for treating or preventing endogenous disorders alleviated by inhibiting dopamine reuptake. Such disorders include, but are not limited to, attention deficit disorder, depression, obesity, Parkinson's disease, tic disorders, and addictive disorders.

Disorders alleviated by inhibiting dopamine reuptake are not limited to the specific disorders described herein, as many types of disorders may manifest from the primary disorder. For example, as disclosed in U.S. Pat. No. 6,132,724 to Blum, attention deficit hyperactivity disorder may manifest itself in the form of alcohol abuse, drug abuse, obsessive compulsive behaviors, learning disorders, reading problems, gambling, manic symptoms, phobias, panic attacks, oppositional defiant behavior, conduct disorder, academic problems in school, smoking, abnormal sexual behaviors, schizoid behaviors, somatization, depression, sleep disorders, general anxiety, stuttering, and tic disorders. All these behaviors and others described herein as associated with disorders alleviated by inhibiting dopamine reuptake are included as disorders as part of this invention. Additionally, clinical terms used herein for many specific disorders are found-in the Quick Reference to the-Diagnostic Criteria From DSM-IV (Diagnostic and Statistical Manual of Mental Disorders, Fourth Edition), The American Psychiatric Association, Washington, D.C., 1994, 358 pages. Specific disorders whose definitions can be found in this reference are described below.

Attention-deficit disorders include, but are not limited to, Attention-Deficit/Hyperactivity Disorder, Predominately Inattentive Type; Attention-Deficit/Hyperactivity Disorder, Predominately Hyperactivity-Impulsive Type; Attention-Deficit/Hyperactivity Disorder, Combined Type; Attention-Deficit/Hyperactivity Disorder not otherwise specified (NOS); Conduct Disorder; Oppositional Defiant Disorder; and Disruptive Behavior Disorder not otherwise specified (NOS).

Depressive disorders include, but are not limited to, Major Depressive Disorder, Recurrent; Dysthymic Disorder; Depressive Disorder not otherwise specified (NOS); and Major Depressive Disorder, Single Episode.

Parkinson's disease includes, but is not limited to, neuroleptic-induced parkinsonism.

Addictive disorders include, but are not limited to, eating disorders, impulse control disorders, alcohol-related disorders, nicotine-related disorders, amphetamine-related disorders, cannabis-related disorders, cocaine-related disorders, hallucinogen use disorders, inhalant-related disorders, and opioid-related disorders, all of which arc further subclassified as listed below.

Eating disorders include, but are not limited to, Bulimia Nervosa, Nonpurging Type; Bulimia Nervosa, Purging Type; and Eating Disorder not otherwise specified (NOS).

Impulse control disorders include, but are not limited to, Intermittent Explosive Disorder, Kleptomania, Pyromania, Pathological Gambling, Trichotillomania, and Impulse Control Disorder not otherwise specified (NOS)

Alcohol-related disorders include, but are not limited to, Alcohol-Induced Psychotic Disorder, with delusions; Alcohol Abuse; Alcohol Intoxication; Alcohol Withdrawal; Alcohol Intoxication Delirium; Alcohol Withdrawal Delirium; Alcohol-Induced Persisting Dementia; Alcohol-Induced Persisting Amnestic Disorder; Alcohol Dependence; Alcohol-Induced Psychotic Disorder, with hallucinations; Alcohol-Induced Mood Disorder; Alcohol-Induced Anxiety Disorder; Alcohol-Induced Sexual Dysfunction; Alcohol-Induced Sleep Disorder; Alcohol-Related Disorder not otherwise specified (NOS); Alcohol Intoxication; and Alcohol Withdrawal.

With respect to alcohol-related disorders, including but not limited to Alcohol Abuse and Alcohol Dependence, (−)-1-(3,4-dichlorophcnyl)-3-azabicyclo[3.1.0]hexane and pharmaceutically acceptable salts thereof can be used to decrease ethanol consumption associated with such alcohol-related disorders. Accordingly, the present invention provides a method for treating or preventing ethanol consumption, comprising administering to a patient in need of such treatment or prevention an effective amount of (−)1-(3,4-dichlorophenyl)-3-azabicyclo[3.1.0]hexane or a pharmaceutically acceptable salt thereof. The present invention also provides a method for treating or preventing ethanol consumption and depression, comprising administering to a patient in need of such treatment or prevention an effective amount of (−)1-(3,4-dichlorophenyl)-3-azabicyclo[3.1.0]hexane or a pharmaceutically acceptable salt thereof. The present invention further provides pharmaceutical compositions for treating or preventing ethanol consumption in a patient comprising an effective amount of (−)-1-(3,4-dichlorophenyl)-3-azabicyclo[3.1.0]hexane or a pharmaceutically acceptable salt thereof. The present invention also provides pharmaceutical compositions for treating or preventing ethanol consumption and depression in a patient comprising (−)-1-(3,4-dichlorophenyl)-3-azabicyclo[3.1.0]hexane or a pharmaceutically acceptable salt thereof.

The routes of administration, dosage amounts and dosage forms described herein can be utilized for the administration of (−)-1-(3,4-dichlorophenyl)-3-azabicyclo[3.1.0]hexane or a pharmaceutically acceptable salt thereof for the prevention or treatment of ethanol consumption or both ethanol consumption and depression to a patient in need of such treatment. Suitable forms of the (−)-1-(3,4-dichlorophenyl)-3-azabicyclo[3.1.0]hexane for use in biologically active compositions and methods of the present invention include its pharmaceutically acceptable salts, polymorphs, solvates, hydrates, and prodrugs.

Administration of an effective amount of (−)-1-(3,4-dichlorophenyl)-3-azabicyclo[3.1.0]hexane or a pharmaceutically acceptable salt thereof, whether alone or in combination with a secondary therapeutic agent, to a patient will detectably treat or prevent ethanol consumption or both ethanol consumption and depression in the patient. In exemplary embodiments, administration of a (−)-1-(3,4-dichlorophenyl)-3-azabicyclo[3.1.0]hexane or a pharmaceutically acceptable salt thereof, whether alone or in combination with a secondary therapeutic agent, to a patient will yield a reduction in ethanol consumption or both ethanol consumption and depression, by at least 10%, 20%, 30%, 50% or greater, up to a 75-90%, or 95% or greater, reduction in ethanol consumption or both ethanol consumption and depression.

Nicotine-related disorders include, but are not limited to, Nicotine Dependence, Nicotine Withdrawal, and Nicotine-Related Disorder not otherwise specified (NOS).

Amphetamine-related disorders include, but are not limited to, Amphetamine Dependence, Amphetamine Abuse, Amphetamine Intoxication, Amphetamine Withdrawal, Amphetamine Intoxication Delirium, Amphetamine-Induced Psychotic Disorder with delusions, Amphetamine-Induced Psychotic Disorders with hallucinations, Amphetamine-Induced Mood Disorder, Amphetamine-Induced Anxiety Disorder, Amphetamine-Induced Sexual Dysfunction, Amphetamine-Induced Sleep Disorder, Amphetamine Related Disorder not otherwise specified (NOS), Amphetamine Intoxication, and Amphetamine Withdrawal.

Cannabis-related disorders include, but are not limited to, Cannabis Dependence; Cannabis Abuse; Cannabis Intoxication; Cannabis Intoxication Delirium; Cannabis-Induced Psychotic Disorder, with delusions; Cannabis-Induced Psychotic Disorder with hallucinations; Cannabis-Induced Anxiety Disorder; Cannabis Related Disorder not otherwise specified (NOS); and Cannabis Intoxication.

Cocaine-related disorders include, but arc not limited to, Cocaine Dependence, Cocaine Abuse, Cocaine Intoxication, Cocaine Withdrawal, Cocaine Intoxication Delirium, Cocaine-Induced Psychotic Disorder with delusions, Cocaine-Induced Psychotic Disorders with hallucinations, Cocaine-Induced Mood Disorder, Cocaine-Induced Anxiety Disorder, Cocaine-Induced Sexual Dysfunction, Cocaine-Induced Sleep Disorder, Cocaine Related Disorder not otherwise specified (NOS), Cocaine Intoxication, and Cocaine Withdrawal.

Hallucinogen-use disorders include, but are not limited to, Hallucinogen Dependence, Hallucinogen Abuse, Hallucinogen Intoxication, Hallucinogen Withdrawal, Hallucinogen Intoxication Delirium, Hallucinogen-Induced Psychotic Disorder with delusions, Hallucinogen-Induced Psychotic Disorders with hallucinations, Hallucinogen-Induced Mood Disorder, Hallucinogen-Induced Anxiety Disorder, Hallucinogen-Induced Sexual Dysfunction, Hallucinogen-Induced Sleep Disorder, Hallucinogen Related Disorder not otherwise specified (NOS), Hallucinogen Intoxication, and Hallucinogen Persisting Perception Disorder (Flashbacks).

Inhalant-related disorders include, but are not limited to, Inhalant Dependence; Inhalant Abuse; Inhalant Intoxication; Inhalant Intoxication Delirium; Inhalant-Induced Psychotic Disorder, with delusions; Inhalant-Induced Psychotic Disorder with hallucinations; Inhalant-Induced Anxiety Disorder; Inhalant Related Disorder not otherwise specified (NOS); and Inhalant Intoxication.

Opioid-related disorders include, but are not limited to, Opioid Dependence, Opioid Abuse, Opioid Intoxication, Opioid Intoxication Delirium, Opioid-Induced Psychotic Disorder, with delusions, Opioid-Induced Psychotic Disorder with hallucinations, Opioid-Induced Anxiety Disorder, Opioid Related Disorder not otherwise specified (NOS), Opioid Intoxication, and Opioid Withdrawal.

Tic disorders include, but are not limited to, Tourette's Disorder, Chronic Motor or Vocal Tic Disorder, Transient Tic Disorder, Tic Disorder not otherwise specified (NOS), Stuttering, Autistic Disorder, and Somatization Disorder.

### 4.4 THERAPEUTIC/PROPHYLACTIC ADMINISTRATION AND COMPOSITION OF THE INVENTION

Due to their activity, (−)-1-(3,4-dichlorophenyl)-3-azabicyclo[3.1.0]hexane and pharmaceutically acceptable salts thereof are advantageously useful in veterinary and human medicine. As described above, (−)-1-(3,4-dichlorophenyl)-3-azabicyclo[3.1.0]hexane and pharmaceutically acceptable salts thereof are useful for the treatment or prevention of a disorder alleviated by inhibiting dopamine reuptake.

When administered to a patient, (−)-1-(3,4-dichlorophenyl)-3-azabicyclo[3.1.0]hexane or a pharmaceutically acceptable salt thereof is typically administered as component of a composition that optionally comprises a pharmaceutically acceptable vehicle. The present compositions, which comprise an effective amount of (−)-1-(3,4-dichlorophenyl)-3-azabicyclo[3.1.0]hexane or a pharmaceutically acceptable salt thereof, are often administered orally. The compositions of the invention can also be administered by any other convenient route, for example, by infusion or bolus injection, by absorption through epithelial or mucocutaneous linings (e.g., oral mucosa, rectal, and intestinal mucosa, etc.) and can be administered together with another biologically active agent. Administration can be systemic or local. Various delivery systems are known, e.g., encapsulation in liposomes, microparticles, microcapsules, and capsules, and can be used to administer (−)-1-(3,4-dichlorophenyl)-3-azabicyclo[3.1.0]hexane and pharmaceutically acceptable salts thereof.

In certain embodiments, the present compositions can comprise (−)-1-(3,4-dichlorophenyl)-3-azabicyclo[3.1.0]hexane and/or one or more pharmaceutically acceptable salts thereof.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of (−)-1-(3,4-dichlorophenyl)-3-azabicyclo[3.1.0]hexane or a pharmaceutically acceptable salt thereof into the bloodstream.

In specific embodiments, it may be desirable to administer (−)-1-(3,4-dichlorophenyl)-3-azabicyclo[3.1.0]hexane or a pharmaceutically acceptable salt thereof locally. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers.

In certain embodiments, it may-be desirable to introduce (−)-1-(3,4-dichlorophenyl)-3-azabicyclo[3.1.0]hexane or a pharmaceutically acceptable salt thereof into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, (−)-1-(3,4-dichlorophenyl)-3-azabicyclo[3.1.0]hexane and pharmaceutically acceptable salts thereof can be formulated as a suppository, with traditional binders and vehicles such as triglycerides.

In another embodiment, (−)-1-(3,4-dichlorophenyl)-3-azabicyclo[3.1.0]hexane and pharmaceutically acceptable salts thereof can be delivered in a vesicle, in particular a liposome (see Langer, 1990, Science 249: 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid, pp. 317-327; see generally ibid).

In yet another embodiment, (−)-1-(3,4-dichlorophenyl)-3-azabicyclo[3.1.0]hexane and pharmaceutically acceptable salts thereof can be delivered in a controlled-release system (see, e.g., Goodson, in Medical Applications of Controlled Release, supra, vol. 2, pp. 115-138 (1984)). Other controlled-release systems discussed in the review by Langer, 1990, Science 249: 1527-1533) may be used. In one embodiment, a pump may be used (see Langer, supra; Sefton, 1987, CRC Crit. Ref. Biamed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macramol. Sci. Rev. Macromal. Chem. 23:61; see also Levy et al., 1985, Science 228: 190; During et al., 1989, Ann. Neurol. 25:351; Howard et al., 1989, J. Neurosurg. 71: 105). In yet another embodiment, a controlled-release system can be placed in proximity of a target of (−)-1-(3,4 dichlorophenyl)-3-azabicyclo[3.1.0]hexane or a pharmaceutically acceptable salt thereof, e.g., the spinal column or brain, thus requiring only a fraction of the systemic dose.

The present compositions can optionally comprise a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, mammals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the pharmaceutically acceptable vehicles are preferably sterile. Water is an exemplary vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in Remington 's Pharmaceutical Sciences, Alfonso R. Gennaro ed., Mack Publishing Co. Easton, Pa., 19th ed., 1995, pp. 1447 to 1676, incorporated herein by reference.

In certain embodiments, (−)-1-(3,4 dichlorophenyl)-3-azabicyclo[3.1.0]hexane or a pharmaceutically acceptable salt thereof is formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharin, cellulose and magnesium carbonate. Such vehicles are preferably of pharmaceutical grade. Typically, compositions for intravenous administration comprise sterile isotonic aqueous buffer. Where necessary, the compositions may also include a solubilizing agent.

In another embodiment, (−)-1-(3,4 dichlorophenyl)-3-azabicyclo[3.1.0]hexane or a pharmaceutically acceptable salt thereof can be formulated for intravenous administration. Compositions for intravenous administration may optionally include a local anesthetic such as lignocaine to lessen pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where (−)-1-(3,4-dichlorophenyl)-3-azabicyclo[3.1.0]hexane or a pharmaceutically acceptable salt thereof is to be administered by infusion, it can be dispensed, for example, with an infusion bottle containing sterile pharmaceutical grade water or saline. Where the (−)-1-(3,4-dichlorophenyl)-3-azabicyclo[3.1.0]hexane or a pharmaceutically acceptable salt thereof is administered by injection, an ampoule of sterile water for injection or saline can be provided so that the ingredients may be mixed prior to administration.

The amount of (−)-1-(3,4-dichlorophenyl)-3-azabicyclo[3.1.0]hexane or a pharmaceutically acceptable salt thereof that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed will also depend on the route of administration, and the seriousness of the disease or disorder, and should be decided according to the judgment of the practitioner and each patient's circumstances. However, suitable dosage ranges for oral administration are generally about 0.001 milligram to about 200 milligrams of (−)-1-(3,4-dichlorophenyl)-3-azabicyclo[3.1.0]hexane or a pharmaceutically acceptable salt thereof per kilogram body weight per day. In specific embodiments of the invention, the oral dose is about 0.01 milligram to about 100 milligrams per kilogram body weight per day, often about 0.1 milligram to about 75 milligrams per kilogram body weight per day, or about 0.5 milligram to about 50 milligrams per kilogram body weight per day, and in certain embodiments about 1 milligram to about 30 milligrams per kilogram body weight per day. In other embodiments, the oral dose is about 1 milligram to about 3 milligrams of (−)-1-(3,4dichlorophenyl)-3-azabicyclo[3.1.0]hexane or a pharmaceutically acceptable salt thereof per kilogram body weight per day. In other embodiments, the oral dose is about 0.1 milligram to about 2 milligrams of (−)-1-(3,4-dichlorophenyl)-3-azabicyclo[3.1.0]hexane or a pharmaceutically acceptable salt thereof per kilogram body weight one to two times per day. The dosage amounts described herein refer to total amounts administered; that is, if (−)-1-(3,4-dichlorophenyl)-3-azabicyclo[3.1.0]hexane and/or one or more pharmaceutically acceptable salts thereof are administered, the preferred dosages correspond to the total amount administered. Oral compositions typically contain about 10% to about 95% active ingredient by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. Suitable dosage ranges for intranasal administration arc generally about 0.01 pg/kg body weight per day to about 1 mg/kg body weight per day. Suppositories generally contain about 0.01 milligram to about 50 milligrams of (−)-1-(3,4-dichlorophenyl)-3-azabicyclo[3.1.0]hexane or a pharmaceutically acceptable salt thereof per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day. Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art.

The invention also provides pharmaceutical packs or kits comprising one or more vessels containing (−)-1-(3,4-dichlorophenyl)-3-azabicyclo[3.1.0]hexane or a pharmaceutically acceptable salt thereof. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains (−)-1-(3,4-dichlorophenyl)-3-azabicyclo[3.1.0]hexane and/or one or more pharmaceutically acceptable salts thereof. In another embodiment, the kit comprises a therapeutic agent and (−)-1-(3,4-dichlorophenyl)-3-azabicyclo[3.1.0]hexane or a pharmaceutically acceptable salt thereof.

(−)-1-(3,4-dichlorophenyl)-3-azabicyclo[3.1.0]hexane and pharmaceutically acceptable salts thereof can be assayed in vitro or in vivo for the desired therapeutic or prophylactic activity prior to use in humans. For example, in vitro assays can be used to determine whether it is preferable to administer (−)-1-(3,4-dichlorophenyl)-3 azabicyclo[3.1.0]hexane, a pharmaceutically acceptable salt thereof, and/or another therapeutic agent. Animal model systems can be used to demonstrate safety and efficacy.

Other methods will be known to the skilled artisan and are within the scope of the invention.

### 4.5 COMBINATION THERAPY

In certain embodiments of the present invention, (−)-1-(3,4-dichlorophenyl)-3-azabicyclo[3.1.0]hexane or a pharmaceutically acceptable salt thereof can be used in combination therapy with at least one other therapeutic agent. (−)-1-(3,4-dichlorophenyl)-3-azabicyclo[3.1.0]hexane or a pharmaceutically acceptable salt thereof and the other therapeutic agent can act additively or otherwise in a complementary fashion, e.g., by lowering side effects elicited by a comparably effective dose of either therapeutically effective agent. In exemplary embodiments, (−)-1-(3,4-dichlorophenyl)-3-azabicyclo[3.1.0]hexane or a pharmaceutically acceptable salt thereof is administered concurrently with the administration of another therapeutic agent, which can be part of the same composition as or in a different composition from that comprising (−)-1-(3,4-dichlorophenyl)-3-azabicyclo[3.1.0]hexane or a pharmaceutically acceptable salt thereof. The other therapeutic agent can be useful for treating and/or preventing (as defined herein) a secondary malady resulting from a disorder alleviated by inhibiting dopamine reuptake. In another embodiment, (−)-1-(3,4 dichlorophenyl)-3-azabicyclo[3.1.0]hexane or a pharmaceutically acceptable salt thereof is administered prior or subsequent to administration of another therapeutic agent. As many of the disorders for which (−)-1-(3,4-dichlorophenyl)-3-azabicyclo[3.1.0]hexane and pharmaceutically acceptable salts thereof are useful in treating are chronic, in one embodiment combination therapy involves alternating between administering a composition comprising (−)-1-(3,4-dichlorophenyl)-3-azabicyclo[3.1.0]hexane or a pharmaceutically acceptable salt thereof and a composition comprising another therapeutic agent. The duration of administration of (−)-1-(3,4-dichlorophenyl)-3-azabicyclo[3.1.0]hexane, a pharmaceutically acceptable salt thereof, or the other therapeutic agent can be, e.g., one month, three months, six months, a year, or for more extended periods, such as the patient's lifetime. In certain embodiments, when (−)-1-(3,4-dichlorophenyl)-3-azabicyclo[3.1.0]hexane or a pharmaceutically acceptable salt thereof is administered concurrently with another therapeutic agent that potentially produces adverse side effects including, but not limited to, toxicity, the other therapeutic agent can advantageously be administered at a dose that falls below the threshold at which the adverse side effect is elicited.

The present invention also includes combinatorial formulations and coordinate administration methods which employ an effective amount of (−)-1-(3,4-dichlorophenyl)-3-azabicyclo[3.1.0]hexane (or a pharmaceutically effective salt, solvate, hydrate, polymorph, or prodrug thereof), and one or more additional active agent(s) that is/are combinatorially formulated or coordinately administered with (−)-1-(3,4-dichlorophenyl)-3-azabicyclo[3.1.0]hexane to yield a combinatorial formulation or coordinate administration method that is effective to prevent or treat ethanol consumption or both ethanol consumption and depression in a patient. Exemplary combinatorial formulations and coordinate treatment methods in this context include, for example, an effective amount of (−)-1-(3,4-dichlorophenyl)-3-azabicyclo[3.1.0]hexane in combination with one or more additional or adjunctive treatment agents or methods for preventing or treating ethanol consumption or both ethanol consumption and depression in a patient, such as one or more anti-alcohol or anti-depressant agent(s) and/or therapeutic method(s).

In related embodiments of the invention, (−)-1-(3,4-dichlorophenyl)-3-azabicyclo[3.1.0]hexane (or a pharmaceutically effective salt, solvate, hydrate, polymorph, or prodrug thereof) can be used in combination therapy with at least one other therapeutic agent or method. In this context, (−)-1-(3,4-dichlorophenyl)-3-azabicyclo[3.1.0]hexane can be administered concurrently or sequentially with administration of a second therapeutic agent, for example a second agent that acts to treat or prevent ethanol consumption or both ethanol consumption and depression or prevent or treat a different disorder or symptom(s) from which (−)-1-(3,4-dichlorophenyl)-3-azabicyclo[3.1.0]hexane is administered. The (−)-1-(3,4-dichlorophenyl)-3-azabicyclo[3.1.0]hexane and the second therapeutic agent can be combined in a single composition or administered in different compositions. The coordinate administration may be done simultaneously or sequentially in either order, and there may be a time period while only one or both (or all) active therapeutic agents, individually and/or collectively, exert their biological activities and therapeutic effects. A distinguishing aspect of all such coordinate treatment methods is that the (−)-1-(3,4-dichlorophenyl)-3-azabicyclo[3.1.0]hexane exerts at least some detectable therapeutic activity towards treating or preventing ethanol consumption or both ethanol consumption and depression, which may or may not be in conjunction with a secondary clinical response provided by the secondary therapeutic agent. Often, the coordinate administration of (−)-1-(3,4-dichlorophenyl)-3-azabicyclo[3.1.0]hexane with a secondary therapeutic agent as contemplated herein will yield an enhanced therapeutic response beyond the therapeutic response elicited by either or both (−)-1-(3,4-dichlorophenyl)-3-azabicyclo[3.1.0]hexane and/or secondary therapeutic agent alone.

Since (−)-1-(3,4-dichlorophenyl)-3-azabicyclo[3.1.0]hexane may need to be administered to a patient chronically for the purpose of preventing or treating ethanol consumption or both ethanol consumption and depression, in one embodiment combination therapy involves alternating between administering (−)-1-(3,4-dichlorophenyl)-3-azabicyclo[3.1.0]hexane (or a pharmaceutically effective salt, solvate, hydrate, polymorph, or prodrug thereof) and a second therapeutic agent (i.e., alternating therapy regimens between the two drugs, e.g., at one week, one month, three month, six month, or one year intervals). Alternating drug regimens in this context will often reduce or even eliminate adverse side effects, such as toxicity, that may attend long-term administration of one or both drugs alone.

The other therapeutic agent can be an anti-attention-deficit-disorder agent. Useful anti-attention-deficit-disorder agents include, but are not limited to, methylphenidate; dextroamphetamine; tricyclic antidepressants, such as imipramine, desipramine, and nortriptyline; and a psychostimulant, such as pemoline and deanol.

The other therapeutic agent can be an anti-addictive-disorder agent. Useful anti-addictive disoder agents include, but are not limited to, tricyclic antidepressants; MAO inhibitors; glutamate antagonists, such as ketamine HCl, dextromethorphan, dextrorphan tartrate and dizocilpine (MK801); degrading enzymes, such as anesthetics and aspartate antagonists; GABA agonists, such as baclofen and muscimol HBr; reuptake blockers; degrading enzyme blockers; glutamate agonists, such as D-cycloserine, carboxyphenylglycine, L-glutamic acid, and cis-piperidinc-2,3-dicarboxylic acid; aspartate agonists; GABA antagonists such as gabazine (SR-95531), saclofen, bicuculline, picrotoxin, and (+) apomorphine HCl; and dopamine antagonists, such as spiperone HCl, haloperidol, and (−) sulpiride.

The other therapeutic agent can be an anti-alcohol agent. As used herein, the term anti-alcohol agent refers to any active drug, formulation or method that exhibits significant activity in mammalian subjects to treat or prevent one or more symtpom(s) of alcohol addiction, alcohol abuse, alcohol intoxication, and/or alcohol withdrawal, including drugs, formulations and methods that significantly reduce, limit, or prevent alcohol consumption in mammalian subjects. Useful anti-alcohol agents in combinatorial formulations and coordinate treatment methods of the invention include, but are not limited to: disulfiram (Litten R Z et al., Development of medications for alcohol use disorders: recent advances and ongoing challenges, Expert Opin Emerg Drugs 10(2):323-43, 2005); naltrexone (Volpicelli, J. R. et al., Naltrexone in the treatment of alcohol dependence, Arch Gen Psychiatry 49:876-880,1992; O'Malley, S. S. et al., Naltrexone and coping skills therapy for alcohol dependence: A controlled study, Arch Gen Psychiatry 49(11):881-887, 1992); acamprosate (Campral) (Swift, R. M. Drug therapy for alcohol dependence, N Engl J Med 340(19):1482-1490, 1999); ondansetron (Zofran®)(Pettinati, H. M. et al., Sertraline treatment for alcohol dependence: Interactive effects of medication and alcohol subtype, Alcohol Clin Exp Res 24(7):1041-1049, 2000; Stoltenberg, Scott F.; Serotonergic Agents and Alcoholism Treatment: A Simulation, Alcoholism: Clinical & Experimental Research 27(12):1853-1859, 2003); sertraline (Zoloft®)(Pettinati, H. M et al., Sertraline treatment for alcohol dependence: Interactive effects of medication and alcohol subtype, Alcohol Clin Exp Res 24(7):1041-1049, 2000); tiapride (Shaw G K et al., Tiapride in the long-term management of alcoholics of anxious or depressive temperament, Br J Psychiatry 150:164-8, 1987); gamma hydroxybutrate,(Alcover) (Poldrugo F. and Addolorato G., The role of g-hydroxybutyric acid in the treatment of alcoholism: from animal to clinical studies. Alcohol Alcoholism 34(1), 15-24, 1999); galanthamine (Novel pharmacotherapies and patents for alcohol abuse and alcoholism 1998-2001, Expert Opinion on Therapeutic Patents, Vol. 11, No. 10, pages 1497-1521 (2001); U.S. Pat. No. 5,932,238); nalmefene (Revex) (Drobes D J et al., Effects of naltrexone and nalmefene on subjective response to alcohol among non-treatment-seeking alcoholics and social drinkers, Alcohol Clin Exp Res., 28(9):1362-70 (2004); naloxone (Julius, D., and Renault, P., eds., Narcotic Antagonists: Naltrexone Progress Report, NIDA Research Monograph Series, Number 9. DHEW Publication No. (ADM) 76-387, Bethesda, Md.: National Institute on Drug Abuse, 1976; Jenab, S., and Inturrisi, C. E., Ethanol and naloxone differentially upregulate delta opioid receptor gene expression in neuroblastoma hybrid (NG108-15) cells, Molecular Brain Research 27:95-102, 1994); desoxypeganine (Doetkotte, R A et al., Effects Of Single And Repeated Administered Desoxypeganine On Ethanol Consumption Of Aa-Rats, Alcoholism: Clinical & Experimental Research, International Society for Biomedical Research on Alcoholism 12th World Congress on Biomedical Alcohol Research, Sep. 29-Oct. 2, 2004, Heidelburg/Mannheim, Germany, 28(8) Supplement:25A, 2004); benzodiazepines (Ntais C et al., Benzodiazepines for alcohol withdrawal, Cochrane Database Syst Rev. (3):CD005063, 2005; Mueller T I et al., Long-term use of benzodiazepines in participants with comorbid anxiety and alcohol use disorders, Alcohol Clin Exp Res. 29(8):1411-8, 2005); neuroleptics such as laevomepromazine (Neurocil®) and thioridazine (Melleril.®); piracetam; clonidine; carbamazepine; clomethiazole (Distraneurin®); levetiracetam; quetiapine (Monnelly E P et al., Quetiapine for treatment of alcohol dependence, J Clin Psychopharmacol. 24(5):532-5, 2004); risperidone; rimonabant; trazodone (Janiri L et al., Adjuvant trazodone in the treatment of alcoholism: an open study, Alcohol 33(4):362-5, 1998); topiramate (Johnson B A et al., Oral topiramate for treatment of alcohol dependence: a randomized controlled trial, Lancet 361:1677-1685, 2003); aripiprazole (Beresford T P et al., Aripiprazole in schizophrenia with cocaine dependence: a pilot study. J Clin Psychopharmacol. 25(4):363-6, 2005); and modafinil (Saletu B et al., On the treatment of the alcoholic organic brain syndrome with an alpha-adrenergic agonist modafinil: double-blind, placebo-controlled clinical, psychomrnetric and neurophysiological studies, Prog Neuropsychopharmacol Biol Psychiatry 14(2):195-214, 1990); amperozide, and modafinil.

The other therapeutic agent can be an anti-nicotine agent. Useful anti-nicotine agents include, but are not limited to, clonidine and bupropion.

The other therapeutic agent can be an anti-opiate agent. Useful anti-opiate agents include, but are not limited to, methadone, clonidine, lofexidine, levomethadyl acetate HCl, naltrexone, and buprenorphine.

The other therapeutic agent can be an anti-cocaine agent. Useful anti-cocaine agents include, but are not limited to, desipramine, amantadine, fluoxidine, and buprenorphine.

The other therapeutic agent can be an appetite suppressant. Useful appetite suppressants include, but are not limited to, fenfluramine, phenylpropanolamine, and mazindol.

The other therapeutic agent can be an anti-lysergic acid diethylamide (“anti-LSD”) agent. Useful anti-LSD agents include, but are not limited to, diazepam.

The other therapeutic agent can be an anti-phencyclidine (“anti-PCP”) agent. Useful anti-PCP agents include, but are not limited to, haloperidol.

The other therapeutic agent can be an anti-Parkinson's-disease agent. Useful anti-Parkinson's-disease agents include, but are not limited to, dopamine precursors, such as levodopa, L-phenylalanine, and L-tyrosine; neuroprotective agents; dopamine agonists; dopamine reuptake inhibitors; anticholinergics such as amantadine and memantine; and 1,3,5-trisubstituted adamantanes, such as 1-amino-3,5-dimethyl-adamantane (U.S. Pat. No.4,122,193 to Sherm et al.).

The other therapeutic agent can be an anti-depression agent. Useful anti-depression agents include, but are not limited to, amitriptyline, clomipramine, doxepine, imipramine, trimipramine, amoxapine, desipramine, maprotiline, nortriptyline, protripylinc, fluoxetine, fluvoxamine, paroxetine, sertraline, venlafaxine, bupropion, nefazodone, trazodone, phenelzine, tranylcypromine, selegiline, clonidine, gabapentin, and 2-pyridinyl[7-(pyridine-4-yl)pyrazolo[1,5-a]pyrimidin-3-yl]methanone compounds having at least one substituent on both the 2- and 4-pyridinyl rings. Useful classes of antidepressant agents include without limitation monoamine oxidase inhibitors, selective serotonin reuptake inhibitors, tricyclic antidepressants, tetracyclic antidepressants, norepinephrine uptake inhibitors, selective norepinephrine reuptake inhibitors, and serotonin and norepinephrine uptake inhibitors.

The other therapeutic agent can be an anxiolytic agent. Useful anxiolytic agents include, but are not limited to, benzodiazepines, such as alprazolam, chlordiazepoxide, clonazepam, clorazepate, diazepam, halazepam, lorazepam, oxazepam, and prazepam; non-benzodiazepine agents, such as buspirone; and tranquilizers, such as barbituates.

The other therapeutic agent can be an antipsychotic drug. Useful antipsychotic drugs include, but are not limited to, phenothiazines, such as chlorpromazine, mesoridazine besylate, thioridazine, acetophenazine maleate, fluphenazine, perphenazine, and trifluoperazine; thioxanthenes, such as chlorprothixene, and thiothixene; and other heterocyclic compounds, such as clozapine, haloperidol, loxapine, molindonc, pimozide, and risperidone. Exemplary anti-psychotic drugs include chlorpromazine HCl, thioridazine HCl, fluphenazine HCl, thiothixene HCl, and molindone HCl.

The other therapeutic agent can be an anti-obesity drug. Useful anti-obesity drugs include, but are not limited, to β-adrenergic receptor agonists, for example β-3 receptor agonists such as, but not limited to, fenfluramine; dexfenfluramine; sibutramine; bupropion; fluoxetine; phentermine; amphetamine; methamphetamine; dextroamphetamine; benzphetamine; phendimetrazine; diethylpropion; mazindol; phenylpropanolamine; norepinephrine; serotonin reuptake inhibitors, such as sibutramine; and pancreatic lipase inhibitors, such as orlistat.

### 5. EXAMPLE

### (−)-1-(3,4-Dichlorophenyl)-3-Azabicyclo [3.1.0]Hexane Hydrochloride

To 279 mg of (±)-1-(3,4-dichlorophenyl)-3-azabicyclo[3.1.0]hexane hydrochloride obtained using the methods described in Epstein et al., J. Med Chem., 24:481-490 (1981) was added 7 mL of 9:1 hexane:isopropyl alcohol, followed by 8 drops of diethylamine. To the resulting mixture was added isopropyl alcohol, dropwise, until a solution was obtained. The solution was concentrated to a volume of 6 mL using a stream of helium gas, and six 1-mL portions of the concentrate-were-subjected to high-performance liquid chromatography using an HPLC instrument equipped with a 1 cm×25 cm Daicel CHIRALPAK AD column (Chiral Technologies, Inc., Exton, Pa.). Elution was carried out at ambient temperature using 95:5 (v/v) hexane:isopropyl alcohol solution containing 0.05% diethylamine as a mobile phase at a flow rate of 6 mL/min. The fraction eluting at about 26.08 to 34 minutes was collected and concentrated to provide a first residue, which was dissolved in a minimal amount of ethyl acetate. Using a stream of nitrogen, the ethyl acetate solution was evaporated to provide a second residue, which was dissolved in 1 mL of diethyl ether. To the diethyl ether solution was added 1 mL diethyl ether saturated with gaseous hydrochloric acid. A precipitate formed, which was filtered, washed with 2 mL of diethyl ether and dried to provide 33 mg of (−)-1-(3,4-dichlorophenyl)3-azabicyclo[3.1.0]hexane hydrochloride of 88% enantiomeric excess. This material was repurified using the chromatography conditions described above. The fraction eluting at about 28 to about 34 minutes was concentrated, acidified, and dried, as described above, to provide 16.0 mg of (−)-1-(3,4-dichlorophenyl)-3-azabicyclo[3.1.0]hexane hydrochloride: optical rotation [α]²⁵_{D}=−56° in methanol at 2 mg/mL; 99.1 % enantiomeric excess.

### 6. EXAMPLE

### Activity Comparison of (−)-1-(3,4-Dichlorophenyl)-3-Azabicyclo[3.1.09 Exane HCL and (+)-1-(3,4-Dichloro Phenyl)-3-Azabicyclo [3.1.0]Hexane HCL in a Dopamine, Norepinephrine, and Serotonin Transporter Binding Assay

Dopamine, norepinephrine, and serotonin uptake-inhibition activity of (−)-1-(3,4-dichlorophenyl)-3-azabicyclo[3.1.0]hexane hydrochloride was compared to that of (±)-1-(3,4-dichlorophenyl)-3-azabicyclo[3.1.0]hexane hydrochloride using a standard dopamine transporter binding assay.

### 6.1 MATERIALS AND METHODS

### 6.1.1 Dopamine Transporter Assay

The dopamine uptake transporter binding assay was performed according to the methods described in Madras et al., 1989, Mol. Pharmacol. 36(4):518-524 and Javitch et al., 1984, Mol. Pharmacol. 26(1):35-44. The receptor source was guinea pig striatal membranes; the radioligand was ³H WIN 35,428 (DuPont-NEN, Boston, Mass.) (60-87 Ci/mmol) at a final ligand concentration of 2.0 nM; the non-specific determinant 1 μM 1-[2-[bis(4-Fluorophenyl)methoxy]ethyl]-4-[3-phenylpropyl]piperazine dihydrochloride (“GBR 12909”), a high-affinity dopamine uptake inhibitor; reference compound was also GBR 12909. (−)-1-(3,4-dichlorophenyl)-3-azabicyclo[3.1.0]hexane HCI was obtained according to the method of Example 5, above. Reactions were carried out in 50 mM TRIS-HCI (pH 7.4), containing 120 mM NaCl and at 0° C. to 4° C. for two hours. The reaction was terminated by rapid vacuum filtration onto glass fiber filters. Radioactivity trapped in the filters was determined and compared to control values in order to ascertain the interactions of the test compound with the dopamine uptake site. The data are reported in Table 1 below.

### 6.1.2 Norepinephrine Transporter Assay

The norepinephrine transporter binding assay was performed according to the methods described in Raisman et al., 1982, Eur. J. Pharmacol. 78:345-351 and Langer et al., 1981, Eur. J. Pharmacol. 72:423. The receptor source was rat forebrain membranes; the radio ligand was [³H]nisoxetine (60-85 Ci/mmol) at a final ligand concentration of 1.0 nM; the non-specific determinant 1 μM desipramine (“DMI”), a high affinity norepinephrine uptake inhibitor; reference compound was desipramine (“DMI”), imipramine, amitriptyline, or nisoxetine. (−)-1-(3,4-dichlorophenyl)-3-azabicyclo[3.1.0]hexane HC1 was obtained according to the method of Example 5, above. Reactions were carried out in 50 mM TRIS-HCI (pH 7.4), containing 300 mM NaCl and 50 mM KCl and at 0° C. to 4° C. for four hours. The reaction was terminated by rapid vacuum filtration onto glass fiber filters. Radioactivity trapped in the filters was determined and compared to control values in order to ascertain the interactions of the test compound with the norepinephrine uptake site. The data are reported in Table 2 below.

### 6.1.3 Serotonin Transporter Assay

The serotonin transporter binding assay was performed according to the methods described in D'Amato et al., 1987, J. Pharmacol. & Exp. Ther. 242:364-371 and Brown et al., 1986, Eur. J. Pharmacol. 123:161-165. The receptor source was human platelet membranes; the radioligand was [³H]citalopram (70-87 Ci/mmol) at a final ligand concentration of 0.7 mM; the non-specific determinant 1 μM clomipramine, a high affinity serotonin uptake inhibitor; reference compound was imipramine. (−)-1-(3,4-dichlorophenyl)-3-azabicyclo[3.1.0]hexane HC1 was obtained according to the method of Example 5, above. Reactions were carried out in 50 mM TRIS-HCl (pH 7.4), containing 120 mM NaCl and 5 mM KCl and at 25° C. for one hour. The reaction was terminated by rapid vacuum filtration onto glass fiber filters. Radioactivity trapped in the filters was determined and compared to control values in order to ascertain the interactions of the test compound with the serotonin uptake site. The data are reported in Table 3 below.

### 6.2 RESULTS

TABLE 1
Dopamine Transporter Binding Assay

| Compound | Kᵢ |
|---|---|
| (−)-1-(3,4-dichlorophenyl)-3-azabicyclo[3.1.0]hexane HCl | 2.61 × 10⁻⁷ |
| (±)-1-(3,4-dichlorophenyl)-3-azabicyclo[3.1.0]hexane HCl | 1.54 × 10⁻⁷ |
| GBR 12909 | 1.16 × 10⁻⁸ |

TABLE 2
Norepinephrine Transporter Binding Assay

| Compound | Kᵢ |
|---|---|
| (−)-1-(3,4-dichlorophenyl)-3-azabicyclo[3.1.0]hexane HCl | N/A |
| (±)-1-(3,4-dichlorophenyl)-3-azabicyclo[3.1.0]hexane HCl | 1.42 × 10⁻⁷ |
| Desimipramine HCl (“DMI”) | 1.13 × 10⁻⁹ |

N/A = no measurable affinity

TABLE 3
Serotonin Transporter Binding Assay

| Compound | Kᵢ |
|---|---|
| (−)-1-(3,4-dichlorophenyl)-3-azabicyclo[3.1.0]hexane HCl | N/A |
| (±)-1-(3,4-dichlorophenyl)-3-azabicyclo[3.1.0]hexane HCl | 1.87 × 10⁻⁷ |
| Imipramine HCl | 2.64 × 10⁻⁸ |

N/A = no measurable affinity

The data in Table 1 show that both (−)-1-(3,4-dichlorophenyl)-3-azabicyclo[3.1.0]hexane HCl and (+)-1-(3,4-dichlorophenyl)-3-azabicyclo[3.1.0]hexane HCI have affinity for the dopamine uptake site. Conversely, the data in Tables 2 and 3 show that the (+)-1-(3,4-dichlorophenyl)-3-azabicyclo[3.1.0]hexane HCl has affinity for the norepinephrine and serotonin uptake sites, wherein the (−)-1-(3,4-dichlorophenyl)-3-azabicyclo[3.1.0]hexane HCl has no measurable affinity for the norepinephrine and serotonin uptake sites. Although the (+)-1-(3,4-dichlorophenyl)-3-azabicyclo[3.1.0]hexane HCl has a higher binding affinity for the dopamine reuptake site than the (−)-1-(3,4-dichlorophenyl)-3-azabicyclo[3.1.0]hexane HCl, the use of the (−)-1-(3,4-dichlorophenyl)-3-azabicyclo[3.1.0]hexane HCl can be more advantageous than the (+)-1-(3,4-dichlorophenyl)-3-azabicyclo[3.1.0]hexane HCl for inhibiting dopamine uptake because of its specificity for inhibiting dopamine uptake. In other words, the use of the (−)-1-(3,4-dichlorophenyl)-3-azabicyclo[3.1.0]hexane HCl can prevent undesirable side effects associated with inhibiting norepinephrine uptake and serotonin uptake, such as hypertension and sexual dysfunction, respectively.

### 7. EXAMPLE

### The Effect of (−)-1-(3,4-Dichlorophenyl)-3-Azabicyclo[3.1.0]Hexane on the Volitional Consumption of Ethanol in the HEP (High Ethanol Preferring) Strain of Rat

### 7.1 MATERIALS AND METHODS

The study described here was performed using procedures modified from previous reports (Myers R D et al., Genetics of alcoholism: Rapid development of a new high ethanol preferring (HEP) strain of female and male rats, Alcohol 16:343-357, 1998; McMillen B A et al., Effects of NMDA glutamate receptor antagonist drugs on the consumption of ethanol by a genetic drinking rat, Brain Res. Bull 64:279-284, 2004). Briefly, subject male rats were taken from the selectively bred Myers' high ethanol preferring (MHEP) line (Myers et al., Alcohol 16:343-357, 1998), which drink copious amounts of ethanol, even in the presence of palatable alternatives. These rats were subjected to an ethanol preference selection protocol, 3% to 30% during a 10-day “step up” procedure that ensures the maximum amount of drinking at each rat's desired ethanol concentration, with the proportion of ethanol to total fluid consumption of approximately 50%.

After the rats were selected and drinking a fixed concentration of ethanol, they were subjected to a three-day baseline ethanol consumption period. This was followed by a three-day period of test drug administration (IP), then a three-day post-treatment period. During this entire study, the following parameters were measured on a daily basis: volume of water consumed; volume of ethanol solution consumed; weight of food consumed; and rat body weight. The test agent, (−)-1-(3,4-dichlorophenyl)-3-azabicyclo[3.1.0]hexane HCl, was administered twice daily in doses of 5, 10 and 20 mg/kg (e.g., 2×5 mg/kg=10 mg/kg/day), with injections occurring at 2 hours before and 2 hours after lights out. Doses were calculated as the free base form of each drug.

Once drinking stabilized to baseline levels post-treatment, a different dose of drug was administered. Some rats did not return to baseline ethanol consumption levels, and were excluded from further investigation. The order of drug doses or vehicle (0.9% saline) was set up in a counter-balanced design. The data were averaged for each period for each rat then grouped and analyzed by one-way repeated measures ANOVA and Tukey's post-hoc analysis.

### 7.2 RESULTS AND DISCUSSION

The administration of (−)-1-(3,4-dichlorophenyl)-3-azabicyclo[3.1.0 ]hexane had pronounced effects on the volitional consumption of ethanol, without a strong anti-caloric effect, in the MHEP rat. Table 4 presents the data for the amount of ethanol consumed during the pre-treatment, treatment and post-treatment periods and shows a strong dose-dependent decrease in consumption by this drug.

TABLE 4
Effect of (−)-1-(3,4-dichlorophenyl)-3-azabicyclo[3.1.0]hexane
treatment on the volitional consumption of ethanol by the male
mHEP rat. Consumption was recorded during a three-day pre-treatment
period, during three-days of b.i.d. injections and then a three-day
post-treatment period.

| | Ethanol Consumption | | |
|---|---|---|---|
| | (g ethanol/kg/day) | | |
| | Mean ± s.e.m. | | |
| Drug mg/kg (n) | pre- | during | post- |
| Vehicle (7) | 6.36 ± 0.54 | 6.25 ± 0.57 | 6.26 ± 0.35 |
| (−)-1-(3,4-dichlorophenyl)-3-azabicyclo[3.1.0]hexane | | | |
| 2.5 (7) | 5.39 ± 0.89 | 4.32 ± 0.69 | 5.79 ± 0.86^{a} |
| 5.0 (8) | 6.00 ± 0.59 | 4.33 ± 0.62* | 5.66 ± 0.64 |
| 10.0 (7) | 6.77 ± 0.51 | 3.50 ± 0.45* | 4.15 ± 0.61* |
| 20.0 (7) | 5.26 ± 0.49 | 1.80 ± 0.28* | 2.89 ± 0.45* |

*different from pre-treatment period, p at least less than 0.05
^{a}post-treatment different from treatment period only, p at least less than 0.05

At the highest dose tested of 20 mg/kg b.i.d., there was a 65.8% decrease in consumption compared to the pre-treatment period that continued to be exhibited during the post-treatment period, which was depressed by 55.1% (F2,6=26.84; p<0.0001). Table 1 shows that the 10 mg/kg b.i.d. dose of DOV 102,677 also had a significant carry over effect during the post-treatment period. The effect was not due to a general effect of reduced fluid consumption, because the proportion of alcohol consumed, that is, the volume of ethanol consumed divided by total volume of ethanol solution and water, also showed a marked decrease during and after the 20 mg/kg b.i.d. (−)-1-(3,4-dichlorophenyl)-3-azabicyclo[3.1.0]hexane treatment of 62.3% and 41%, respectively (F2,6=22.68; p<0.0001) (Table 5).

TABLE 5
Effect of (−)-1-(3,4-dichlorophenyl)-3-azabicyclo[3.1.0]hexane
on the proportion of ethanol to total fluids consumed by the
male mHEP rat. Consumption was recorded during a three-day
pre-treatment period, during three-days of b.i.d. injections
and then a three-day post-treatment period.

| | Relative Fluid Consumption | | |
|---|---|---|---|
| | ml ethanol/(ml ethanol + ml water) | | |
| | Mean ± s.e.m. | | |
| Drug mg/kg (n) | pre- | during | post- |
| Vehicle (7) | 0.71 ± 0.04 | 0.68 ± 0.04 | 0.68 ± 0.05 |
| (−)-1-(3,4-dichlorophenyl)-3-azabicyclo[3.1.0]hexane | | | |
| 2.5 (7) | 0.54 ± 0.03 | 0.44 ± 0.05 | 0.56 ± 0.07 |
| 5.0 (8) | 0.63 ± 0.05 | 0.48 ± 0.05* | 0.61 ± 0.04 |
| 10.0 (7) | 0.62 ± 0.02 | 0.30 ± 0.04* | 0.46 ± 0.09 |
| 20.0 (6) | 0.61 ± 0.07 | 0.23 ± 0.04* | 0.36 ± 0.08* |

*different from pre-treatment period, p at least less than 0.05

For studies of drug effects on ethanol consumption, it is important to measure food intake since ethanol represents calories to the animal. None of the doses of (−)-1-(3,4-dichlorophenyl)-3-azabicyclo[3.1.0]hexane altered food intake during the treatment period. During the post-treatment period, the rats that had received the 10 and 20 mg/kg b.i.d. doses showed statistically significant (F2,6=11.49; p<0.01 and F2,6=5.10; p<0.05, respectively) increases in food intake as compared to the pre-treatment period. This means the ability of (−)-1-(3,4-dichlorophenyl)-3-azabicyclo[3.1.0]hexane to reduce alcohol consumption is not simply the result of a corresponding decrease in caloric intake.

TABLE 6
Effect of (−)-1-(3,4-dichlorophenyl)-3-azabicyclo[3.1.0]hexane
treatment on the consumption of food by the male mHEP rat.
Consumption was recorded during a three-day pre-treatment
period, during three-days of b.i.d. injections and then
a three-day post-treatment period.

| | Food Consumption | | |
|---|---|---|---|
| | g food/day | | |
| | Mean ± s.e.m. | | |
| Drug mg/kg (n) | pre- | during | post- |
| Vehicle (7) | 18.4 ± 0.8 | 17.9 ± 0.6 | 17.9 ± 0.8 |

(−)-1-(3,4-dichlorophenyl)-3-azabicyclo[3.1.0]hexane treatment

| | | | |
|---|---|---|---|
| 2.5 (7) | 19.5 ± 1.0 | 19.6 ± 0.8 | 19.9 ± 1.1 |
| 5.0 (8) | 18.7 ± 0.5 | 18.4 ± 0.6 | 19.9 ± 0.7 |
| 10.0 (7) | 19.4 ± 1.2 | 18.1 ± 1.2 | 22.0 ± 1.8^{a} |
| 20.0 (7) | 18.4 ± 1.8 | 19.3 ± 1.1 | 21.6 ± 1.5* |

*different from pre-treatment period, p at least less than 0.05
^{a}post-treatment different from pre-treatment and during treatment period, p at least less
than 0.05

The rats treated with (−)-1-(3,4-dichlorophenyl)-3-azabicyclo[3.1.0]hexane showed small increases in body weight expected by rats of this age over these short periods of time.

TABLE 7
Effect of (−)-1-(3,4-dichlorophenyl)-3-azabicyclo[3.1.0]hexane
on the body weight of the male mHEP rat. Consumption was recorded
during a three-day pre-treatment period, during three-days of b.i.d.
injections and then a three-day post-treatment period.

| | Bodyweight, (kg) | | |
|---|---|---|---|
| | Mean ± s.e.m. | | |
| Drug mg/kg (n) | pre- | during | post- |
| Vehicle (7) | 0.392 ± 0.013 | 0.396 ± 0.013* | 0.398 ± 0.013* |

(−)-1-(3,4-dichlorophenyl)-3-azabicyclo[3.1.0]hexane

| | | | |
|---|---|---|---|
| 2.5 (7) | 0.369 ± 0.019 | 0.372 ± 0.019* | 0.373 ± 0.019* |
| 5.0 (8) | 0.392 ± 0.009 | 0.394 ± 0.009 | 0.394 ± 0.007 |
| 10.0 (7) | 0.381 ± 0.028 | 0.381 ± 0.028 | 0.382 ± 0.028 |
| 20.0 (7) | 0.421 ± 0.015 | 0.423 ± 0.015 | 0.418 ± 0.015^{a} |

*different from pre-treatment period, p at least less than 0.05
^{a}post-treatment different from treatment period only, p at least less than 0.05

These data demonstrate that over the dose range of 5.0 to 20 mg/kg b.i.d., (−)-1-(3,4-dichlorophenyl)-3-azabicyclo[3.1.0]hexane significantly reduced the volitional consumption of ethanol with little change in food consumption. This latter result indicates that the effect is a pharmacological interaction, not an anti-caloric or appetitive effect. It should be noted that (−)-1-(3,4-dichlorophenyl)-3-azabicyclo[3.1.0]hexane produced a strong decrease in the proportion of ethanol consumed, which indicates that the rats were still drinking water. Thus, the effect is not due to an adipsic action by the drug.

These results demonstrate that (−)-1-(3,4-dichlorophenyl)-3-azabicyclo[3.1.0]hexane, which has anti-depressant properties, has strong effects on the volitional selection of ethanol in a free choice paradigm. This is a naturalistic paradigm since ethanol is available for 24 hr. per day, and was simply mixed with tap water, in the absence of a masking agent to hide the taste, and appears to have face validity for the human condition.

### 8. EXAMPLE

### Activity Comparison of (−)-1-(3,4-Dichlorophenyl)-3-Azabicyclo[3.1.0]Hexane HCL (+)-1-(3,4-Dichlorophenyl)-3-Azabicyclo [3.1.0]Hexane HCL and (±)-1-(3,4-Dichlorophenyl)-3-Azabicyclo[3.1.0]Hexane HCL in Dopamine, Norepinephrine, and Serotonin Transporter Binding and Uptake Assays Using Recombinant Human Receptors

Dopamine, norepinephrine, and serotonin uptake-inhibition activity of (−)-1-(3,4-dichlorophenyl)-3-azabicyclo[3.1.0]hexane hydrochloride was compared to that of (±)-1-(3,4-dichlorophenyl)-3-azabicyclo[3.1.0]hexane hydrochloride and (+)-1-(3,4-dichlorophenyl)-3-azabicyclo[3.1.0]hexane hydrochloride using human cell lines transfected with cDNA molecules expressing recombinant human receptors.

### 8.1 MATERIALS AND METHODS

### 8.1.1 Materials

Radiolabeled neurotransmitters ([³H]DA, [³H]5-HT, [³H]NE and [¹²⁵I]RTI-55) were purchased from NEN-Life Sciences (Boston, Mass.). Most other reagents were purchased from Sigma Chemical Co. (St. Louis, Mo.). The cloning and characterization of hDAT cDNA used in these experiments (pcDNA1-hDAT) was performed as described previously (Eshelman A J et al., Release of dopamine via the human transporter, Mol. Pharmacol. 45:312-316, 1994; Eshelman A J et al., Characterization of recombinant human dopamine in multiple cell lines, J. Pharmacol. Exp. Ther. 274:276-283 (1995). The cDNA for the hSERT (Ramamoorthy, S et al., Antidepressant- and cocaine-sensitive human serotonin transporter: Molecular cloning, expression, and chromosomal localization, Proc. Natl. Acad. Sci. USA 90:2542-2546 (1993)), and HEK cells expressing the hNET (HEK-hNET)(Galli A et al., Sodium-dependent norepinephrine-induced currents in norepinephrine-transporter transfected HEK-293 cells blocked by cocaine and antidepressants, J. Exp. Biol. 198:2197-2212 (1995) were used as previously described.

### 8.1.2 Binding Assays

HEK-hDAT and HEK-hSERT cells were incubated in Dulbecco's modified Eagle's medium supplemented with 5% fetal bovine serum, 5% calf bovine serum, 0.05 U penicillin/streptomycin, and puromycin (2 μg/ml). HEK-hNET cells were incubated in Dulbecco's modified Eagle's medium supplemented with 10% fetal bovine serum, 0.05 U penicillin/streptomycin, and geneticin (300 μg/ml). Cells were grown until confluent on 150-mm-diameter tissue culture dishes in a humidified 10% CO₂ environment at 37° C. Medium was removed from the plates, cells washed with 10 ml of PBS, lysis buffer (10 ml, 2 mM HEPES, 1 mM EDTA) was added, and plates were placed on ice for 10 min. Cells were scraped from plates and centrifuged for 20 min at 30,000 g. The pellet was resuspended in 6 to 24 ml of 0.32 M sucrose with a Polytron homogenizer at setting 7 for 5 sec.

Assays contained an aliquot of membrane preparation (approximately 12-30 μg protein, depending on the cell line, which resulted in binding <10% of the total radioactivity), drug, [¹²⁵I]RTI-55 (40-80 μM final concentration) in a final volume of 250 μl. Krebs-HEPES assay buffer (25 mM HEPES, 122 mM NaCl, 5 mM KCl, 1.2 mM MgSO₄, 2.5 mM CaCl₂, 1 μM pargyline, 100 μM tropolone, 2 mg glucose/ml, 0.2 mg ascorbic acid/ml, pH 7.4) was used for all assays. Specific binding was defined as the difference in binding observed in the presence and absence of 5 μM mazindol (HEK-hDAT and -NET) or 5 μM imipramine (HEK-hSERT). Membranes were preincubated with drugs at room temperature for 10 min before the addition of [¹²⁵I]RTI-55, unless indicated otherwise. The reaction was incubated for 90 min at room temperature in the dark and was terminated by filtration through Wallace Filtermat A filters using a 96-well Tomtec cell harvester. Scintillation fluid (50 μl) was added to each filtered spot, and radioactivity remaining on the filter was determined using a Wallace 1205 Betaplate or 1405 microBeta scintillation counter. Competition experiments were conducted with duplicate determinations for each point.

### 8.1.3 Inhibition of Radiolabeled Neurotransmitter Uptake in HEK-hDAT, -hSERT and -hNET Cells

Cells were grown on 150-mm-diameter tissue culture plates as described above. Medium was removed and plates were washed twice with Ca²⁺, Mg²⁺-free PBS. Fresh Ca²⁺, Mg²⁺-free PBS (2.5 ml) was then added to each plate and plates were placed in a 25° C. water bath for 5 min. Cells were gently scraped from plates, and cell clusters were separated by trituration with a pipette for 5 to 10 aspirations and ejections.

Aliquots (50 μl) of the suspended cells were added to assay tubes containing drugs and Krebs-HEPES assay buffer in a final volume of 0.5 ml. Competition experiments were conducted with triplicate determinations at each point. After a 10-min preincubation in a 25° C. water bath (unless indicated otherwise), [³H]neurotransmitter (20 nM final concentration; [³H]DA, [³H]5-HT, or [³H]NE; 56, 26.9, or 60 Ci/mmol, respectively) was added, and the assay was incubated for 10 min. The reaction was terminated by filtration through Wallace Filtermat A filters, presoaked in 0.05% polyethylenimine, using a Tomtec cell harvester. Scintillation fluid was added to each filtered spot, and radioactivity remaining on the filters was determined as described above. Specific uptake was defined as the difference in uptake observed in the absence and presence of 5 μM mazindol (HEK-hDAT and -NET) or 5 μM imipramine (HEK-hSERT).

### 8.1.4 Data Analysis

Prism software (GraphPad Software, San Diego, Calif.) was used to analyze all kinetic, saturation, and competition binding data. IC₅₀ values were converted to Kᵢ values using the Cheng-Prusoff equation (Cheng Y and Prusoff W H, Relationship between the inhibition constant (Kᵢ) and the concentration of inhibitor which causes 50-per cent binding inhibition (I₅₀) of an enzymatic reaction, Biochem Pharmacol 22: 3099-3108 (1973)).

### 8.2 RESULTS AND DISCUSSION

The results of this experiment are summarized in Table 8.

TABLE 8
Activity comparison of (−)-1-(3,4-dlchlorophenyl)-3-azabicyclo[3.1.0]hexane
HCl, (+)-1-(3,4-dichlorophenyl)-3-azabicyclo[3.1.0]hexane HCl and (±)-1-(3,4-
dichlorophenyl)-3-azabicyclo[3.1.0]hexane HCl in dopamine, norepinephrine, and
serotonin transporter binding and uptake assays using recombinant human receptors.

| | Dopamine | | Serotonin | | Norepinephrine | |
|---|---|---|---|---|---|---|
| | Binding | Uptake | Binding | Uptake | Binding | Uptake |
| Compound | Kᵢ, nM | IC₅₀, nM | Kᵢ, nM | IC₅₀, nM | Kᵢ, nM | IC₅₀, nM |
| Racemate | 186 ± 40 | 78 ± 15 | 188 ± 28 | 13.8 ± 1.5 | 378 ± 43 | 20.3 ± 6.1 |
| (+)-Enantiomer | 213 ± 56 | 96 ± 20 | 99 ± 16 | 12.3 ± 2.8 | 262 ± 41 | 22.8 ± 3.3 |
| (−)-Enantiomer | 222 ± 43 | 129 ± 15 | 740 ± 140 | 133 ± 26 | 1030 ± 76 | 103 ± 27 |

The data in Table 8 show that both (−)-1-(3,4-dichlorophenyl)-3-azabicyclo[3.1.0]hexane HCl, (+)-1-(3,4-dichlorophenyl)-3-azabicyclo[3.1.0]hexane HCI and (±)-1-(3,4-dichlorophenyl)-3-azabicyclo[3.1.0]hexane HCl have similar affinities for the dopamine transporter as measured by binding and uptake. Conversely, the data in Table 8 show that (+)-1-(3,4-dichlorophenyl)-3-azabicyclo[3.1.0]hexane HCl and (±)-1-(3,4-dichlorophenyl)-3-azabicyclo[3.1.0]hexane HCl have substantially greater affinity for the serotonin and norepinephrine transporters than (−)-1-(3,4-dichlorophenyl)-3-azabicyclo[3.1.0]hexane HCl as measured by both binding and reuptake. With respect to binding to the serotonin transporter, there is a 3.9-fold difference in affinity between (−)-enantiomer and the racemate, and a 7.4 fold difference in affinity between the (−)- and (+)-enantiomers of this compound. With respect to binding to the norepinephrine transporter, there is a 2.7-fold difference in affinity between (−)-enantiomer and the racemate, and a 3.9-fold difference in affinity between (−)- and (+)-enantiomers. With respect to uptake at the serotonin transporter, there is a 9.6-fold difference in affinity between the (−)-enantiomer and the racemate, and a 1 0-fold difference in affinity between (−)- and (+)-enantiomers. With respect to the inhibition of norepinephrine uptake , there is a 5.0-fold difference in potency between (−)-enantiomer and the racemate, and a 4.5-fold difference in potency between (−)-1-(3,4-dichlorophenyl)-3-azabicyclo[3.1.0]hexane HCl and (+)-1-(3,4-dichlorophenyl)-3-azabicyclo[3.1.0]hexane HCl.

These results are consistent with the results set forth in Example 6 above obtained using a different model system. In particular, even though the model system derived from human materials used in this study shows that (−)-1-(3,4-dichlorophenyl)-3-azabicyclo[3.1.0]hexane HCl has some affinity for and potency in blocking neurotransmitter uptake by recombinant human serotonin and norepinephrine receptors, it is significantly less than that of (±)-1-(3,4-dichlorophenyl)-3-azabicyclo[3.1.0]hexane HCl and (+)-1-(3,4-dichlorophenyl)-3-azabicyclo[3.1.0]hexane HCl. The profile of inhibition of monoamine neurotransmitter transporters would be expected to provide a novel combination of therapeutic indications and reduced side effects compared to inhibitors of either serotonin or norepinephrine uptake alone.

Successful inhibition of dopamine reuptake has been has been associated with the treatment of attention deficit disorder, depression, obesity, Parkinson's disease, a tic disorder and an addictive disorder (Hitri et al., 1994, Clin. Pharmacol. 17: 1-22; Noble, 1994, Alcohol Supp. 2:35-43; and Blum et al., 1995, Pharmacogenetics 5:121-141). Because of its specificity for inhibiting dopamine uptake, (−)-1-(3,4-dichlorophenyl)-3-azabicyclo[3.1.0]hexane or a pharmaceutically acceptable salt thereof will be more advantageous than (±)-1-(3,4-dichlorophenyl)-3-azabicyclo[3.1.0]hexane or a pharmaceutically acceptable salt thereof in treating or preventing a disorder alleviated by inhibiting dopamine reuptake in a patient.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

Although the foregoing invention has been described in detail by way of example for purposes of clarity of understanding, persons of ordinary skill in the art will understand that certain changes and modifications may be practiced within the scope of the appended claims which are presented by way of illustration not limitation. In this context, the invention is not limited to the particular formulations, processes, and materials disclosed herein, as such formulations, process steps, and materials may vary somewhat. Also, the terminology employed herein is used for describing particular embodiments only, and is not intended to be limiting of the invention embodied in the claims. Various publications and other reference information have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

1-56. (canceled)

57. A method for treating or preventing an alcohol-related disorder in a mammalian subject consumption, comprising administering to the subject an anti-alcohol effective amount of (−)1-(3,4-dichlorophenyl)-3-azabicyclo[3.1.0]hexane or a pharmaceutically acceptable salt thereof.

58. The method according to claim 57, wherein the (−)-1-(3,4-dichlorophenyl)-3-azabicyclo[3.1.0]hexane or pharmaceutically acceptable salt thereof is administered in a formulation that comprises no more than about 2% w/w of the corresponding (+)-enantiomer.

59. The method according to claim 57, wherein the (−)-1-(3,4-dichlorophenyl)-3-azabicyclo[3.1.0]hexane or pharmaceutically acceptable salt thereof is administered in a formulation that comprises no more than about 1% w/w of the corresponding (+)-enantiomer.

60. The method according to claim 57, wherein said anti-alcohol agent is effective to reduce alcohol consumption by said subject in comparison to a control subject that does not receive said anti-alcohol agent.

61. The method according to claim 57 further comprising administering a second anti-alcohol agent to said subject.

62. A pharmaceutical composition comprising an anti-alcohol effective amount of (−)-1-(3,4-dichlorophenyl)-3-azabicyclo[3.1.0]hexane or a pharmaceutically acceptable salt thereof in a formulation that is substantially free of a corresponding (+)-enantiomer of 1-(3,4-dichlorophenyl)-3-azabicyclo[3.1.0]hexane.

63. A composition according to claim 62, wherein said formulation comprises no more than about 2% w/w of the corresponding (+)-enantiomer.

64. A composition according to claim 62, wherein said formulation comprises no more than about 1% w/w of the corresponding (+)-enantiomer.

65. A composition according to claim 62 further comprising a second anti-alcohol agent.

66. The method of claim 61, wherein said second anti-alcohol agent is selected from the group consisting of: disulfiram; naltrexone; acamprosate; ondansetron; sertraline; galanthamine; nalmefene; naloxone; desoxypeganine; benzodiazepines; neuroleptics; risperidone; rimonabant; trazodone; topiramate; and aripiprazole.

67. A pharmaceutical composition according to claim 65, wherein said second anti-alcohol agent is selected from the group consisting of: disulfiram; naltrexone; acamprosate; ondansetron; sertraline; galanthamine; nalmefene; naloxone; desoxypeganine; benzodiazepines; neuroleptics; risperidone; rimonabant; trazodone; topiramate; and aripiprazole
